(19) United States
(12) Patent Application Publication
(10) Pub. No.: US 2015/0238394 A1
(43) Pub. Date: Aug. 27, 2015

(54) Triggerable Compositions For Two-Stage, Controlled Release of Proactive Chemistry Using Aldehydes

(71) Applicant: Kimberly-Clark Worldwide, Inc., Neenah, WI (US)

(72) Inventors: Xuedong Song, Alpharetta, GA (US); Yiming Weng, Alpharetta, GA (US)

(73) Assignee: Kimberly-Clark Worldwide, Inc., Neenah, WI (US)

(21) Appl. No.: 14/192,116

(22) Filed: Feb. 27, 2014

(51) Int. Cl. A61K 8/11; B01J 20/22; A61K 8/81; C11B 9/00; A61K 8/41; A61Q 19/00

(52) U.S. Cl. CPC A61K 8/11; A61K 8/416; A61Q 19/00; A61K 8/8152; A61K 8/8182; C11B 9/0015; B01J 20/22; A61K 2800/56

(57) ABSTRACT

A triggerable composition for two-stage, controlled release of a functional active chemical includes an aldehyde-based functional active incorporated in an organic salt-based acetal that releases the functional active through a hydrolysis reaction upon contact with an aqueous medium, and an encapsulation material for encapsulating the organic salt-based acetal, the encapsulation material triggerable to release or expose the organic salt-based acetal upon the occurrence of an environmental stimulus such as a pH change. The triggerable composition can be included in an absorbent article having at least one absorbent layer or in a viscous liquid such as a lotion, cream, or medicament.

### BACKGROUND

The present disclosure pertains to a composition that controls the chemical release of functionally active components from a previously inactive and modified state. In particular, the present disclosure pertains to a composition that gradually or rapidly releases active chemical components upon the occurrence of specific environmental stimuli. The composition can be used in bandages, hygiene products, health care products and skin-contacting beauty products, as well as in consumer product applications. The present disclosure also relates to such bandages, hygiene products, health care products, beauty products and consumer products incorporating such chemistry.

A large number of functionally active chemicals are known for use with personal care and beauty products, hygiene products, health care related products, and skin-contacting products. For example, such actives include antimicrobial or antibacterial agents, antioxidant agents, antiseptic-type agents, skin-repairing agents, and fragrances. Unfortunately, many of these functionally-active chemicals are not stable or do not have ideal properties under various environmental conditions. For example, if such actives include volatile components such as those found in fragrances, they can dissipate into the surrounding environment upon exposure to air and humidity conditions. Therefore, such chemicals can demonstrate short shelf lives when in use, and can present serious packaging/storage concerns. As a result, costly packaging can be necessary for products incorporating such chemicals. This instability creates a significant limitation on the wide adoption of the potentially useful chemistry and limits the long-term efficacy of products incorporating such chemistry. Further, processing challenges such as elevated temperatures can exist, and, as a result, can present a need to limit exposure to environmental stimuli during manufacture.

Additional challenges presented by the use of such active chemicals include the difficulties involved with gradually controlling the release of such active chemicals, as well as the potential side effects and costs resulting from use of chemically degraded products. Other actives, such as antioxidants, are also often not stable when exposed to ambient conditions, such as the air of a user's pantry or storage closets. Antioxidants can readily be oxidized by oxygen in the air. Some skin-repairing chemicals are also not stable when exposed to the surrounding environment. For example, the skin-repairing agent retinol is not stable under ambient conditions without protection from the environment. In fact, it can become a skin irritant when its concentration is relatively high. Currently no proactive technology has emerged to be very effective to achieve both property modification and at the same time release-on-demand under mild conditions. For examples, anti-oxidants such as vitamin C and vitamin A are often stabilized through the ester forms which are hydrolyzed into the active forms through enzymes when digested into bodies. In many cases, a large portion of the actives are wasted because they are not hydrolyzed and released to the desired locations. A need therefore exists for a versatile composition that effectively stabilizes functional chemical actives, and releases such actives upon demand, at a desirable rate and profile.

Attempts have been made to overcome the stability and storage limitations presented by such actives. For example, some have suggested stabilizing retinol by encapsulating it in pH-sensitive polymers and then releasing it at a later time by changing the solubility of the encapsulating matrix through a pH change. The encapsulated retinol still suffers significant degradation, presumably from oxidation. Others have suggested converting retinol into an ester as a proactive (a precursor to the retinol active), and then at a later time converting the ester into the active form by use of enzymes present in a user's body after delivery through a user's skin. With such methodology, however, only a small portion of the ester is used effectively by the skin layer and a majority of the esters are wasted by the system. Such a system can also actually lead to side effects when too much retinol ester is used to achieve effective dosages on the skin. Therefore, a need still exists for delivery compositions for skin-repair actives.

In connection with the delivery of fragrances (such as in connection with personal care absorbent products), it has been suggested to encapsulate fragrances in polymeric matrices for stabilization and delivery benefits. Even with such encapsulation technology, however, there is a further need for fragrance encapsulation technology that offers effective protection for such volatiles as well as a controlled release. Existing encapsulation chemistries for consumer products often leak or release prematurely. A continuing need exists for a material composition that both provides stability for unstable actives, and that provides for release of actives in a controlled manner.

### SUMMARY

Here we disclose a new class of acetal-based pro-actives that can be used to modify and improve properties of aldehyde-based actives and release those actives upon contact with samples containing liquid water, such as body secretions. The disclosure also discloses a class of materials that contains the pro-actives and encapsulation materials to achieve controlled release through multiple triggers. Furthermore, the disclosure also discloses products such as personal care absorbent products and family care tissues containing the proactives and related materials.

The current disclosure is directed to a triggerable composition for creating a stable, controlled-release of functional chemical active components using a two-stage release mechanism. The triggerable composition allows for property modification of the functional actives and protection from the surrounding environment, as well as the selective release of such actives upon the occurrence of two select environmental stimuli. The protection and stabilization of the functional active are accomplished through converting an aldehyde-based functional active into an organic salt-based acetal, as well as the incorporation of the organic salt-based acetal functional active into an encapsulation polymer matrix. The triggered release of the functional active from the organic salt-based acetal is dependent upon preselected properties of the encapsulation polymer matrix (first stage trigger), as well as the hydrolysis of the organic salt-based acetal (by an aqueous medium, in a second stage trigger) once the organic salt-based acetal is released or freed from the encapsulation polymer matrix. For the purposes of this application, the term “aqueous medium” means a medium containing “liquid” water as opposed to water vapor. Such aqueous medium is exemplified by but not limited to urine, sweat, vaginal fluids, mucous, menses, and runny, liquid, and loose bowel movements.

The functional active chemicals can be a fragrance, a skin-repairing agent, an antioxidant agent, an antimicrobial/antibacterial agent, an antifungal agent, a hormone, and a medically active agent. Stabilization of the functional active chemicals through an organic salt-based acetal and within an encapsulation polymer matrix prevents the premature release of the chemicals either into the environment or to a desired location. The functional active chemicals can be derived from substances including at least one aldehyde group that are volatile, water-sensitive, unstable, poor solubility, poor skin permeability or easily oxidized by oxygen. Stabilization is accomplished by the incorporation of a radical form of the functional active chemicals into the organic salt-based acetal. The organic salt-based acetal can be readily hydrolyzed upon exposure to an aqueous medium to release the active. The encapsulation polymer matrix protecting the organic salt-based acetal can be designed to be sensitive to water in either a neutral, acidic, or basic condition. The encapsulation polymer matrix protecting the organic salt-based acetal can also be designed to be sensitive to enzymes, ions, or ligands.

In one aspect of the disclosure, a triggerable composition for two-stage, controlled release of a functional active chemical includes an aldehyde-based functional active incorporated in an organic salt-based acetal that releases the functional active through a hydrolysis reaction upon contact with an aqueous medium, and an encapsulation material for encapsulating the organic salt-based acetal, the encapsulation material triggerable to release or expose the organic salt-based acetal upon the occurrence of an environmental stimulus.

In an alternative aspect of the disclosure, an absorbent article includes at least one absorbent layer, the absorbent article including a triggerable composition for two-stage, controlled release of a functional active chemical, the triggerable composition including an organic salt-based acetal of a functional active with at least one aldehyde group configured to release the functional active through a hydrolysis reaction upon contact with an aqueous medium. The triggerable composition also includes an encapsulation material for encapsulating the organic salt-based acetal including a functional active, the encapsulation material triggerable to release or expose the organic salt-based acetal upon the occurrence of an environmental stimulus.

In still another alternative aspect of the disclosure, a triggerable composition for two-stage, controlled release of a functional active chemicals includes an organic salt-based acetal for release of a functional active contained on the organic salt-based acetal, through a hydrolysis reaction upon contact with an aqueous medium, and an encapsulation material for encapsulating the organic salt-based acetal including a functional active, the encapsulation material triggerable to release or expose the organic salt-based acetal upon the occurrence of an environmental stimulus, wherein the environmental stimulus is a pH change.

In another alternative aspect, a viscous liquid includes a triggerable composition for two-stage, controlled release of a functional active chemical, the composition including an organic salt-based acetal of a functional active with at least one aldehyde group configured to release the functional active through a hydrolysis reaction upon contact with an aqueous medium. The composition also includes an encapsulation material for encapsulating the organic salt-based acetal including a functional active, the encapsulation material triggerable to release or expose the organic salt-based acetal upon the occurrence of an environmental stimulus, wherein the viscous liquid is a lotion, cream, or medicament.

Other features and aspects of the present disclosure are discussed in greater detail below.

### DETAILED DESCRIPTION

Stabilization of an aldehyde-based active chemistry and/or modification of its physical and/or chemical property is disclosed herein. The actives have one or more aldehyde groups. The actives can be volatile, water-sensitive, or unstable, or can have poor solubility or poor skin permeability. The stabilization is accomplished by converting the aldehyde-based active into an organic salt-based acetal, which is either semi-solid or solid. The acetal can be hydrolyzed upon exposure to moisture to release the aldehyde-based active. These salt-based acetals contain at least one charged group/counter ion, which improve water-solubility and reduce volatility.

Protection/Controlled triggered-release of the organic salt-based acetal proactive in another stimuli-sensitive encapsulation polymer matrix, which is sensitive to water, or pH, or enzymes. Specific examples of this polymer matrix are provided (Eudragit S-100, Eudragit E-100, and PVP/VA 1-335) though other polymers are contemplated. Eudragit S-100 is an anionic copolymer based on methacrylic acid and methyl methacrylate, and is sensitive to basic aqueous solutions. Eudragit S-100 is insoluble in neutral water but soluble in basic aqueous solution. Eudragit E-100 has amine groups and is not soluble in basic aqueous solution, but soluble in acidic aqueous solutions. PVP/VA 1-335 is a vinylpyrrolidone/vinylacetate copolymer and is soluble in aqueous solutions.

Thus, protection of the aldehyde-based active is accomplished through conversion into a salt-based acetal chemistry, as well as incorporation of the acetal in an encapsulation polymer matrix. The triggered release depends on the properties of the encapsulation polymer matrix, as well as the hydrolysis of the acetal once released from the polymer matrix.

Examples of actives include fragrances, antimicrobial agents, skincare actives, antibiotics, and hormones.

In general, the present disclosure is directed to a composition that includes an encapsulation chemistry for selectively releasing a functional active through an organic salt-based acetal and stimuli-sensitive encapsulation chemistries. The selective triggering of the encapsulation chemistries will expose the organic salt-based acetal to an aqueous medium. In a second stage, upon exposure of the organic salt-based acetal to the aqueous medium, a hydrolysis reaction will occur, resulting in the release of the functional aldehyde active from the organic salt-based acetal into the surrounding environment or at a targeted location. The surrounding environment or targeted location can be onto a user's skin, or into the structure of an article containing the triggerable composition. Such article can be, for example, a health care product such as a garment or bandage, a hygiene product such as a tissue or wipe, a skin-contacting beauty product such as a facial wrap, an absorbent consumer/personal care article such as a feminine care pad or liner, a baby or child care diaper, or an adult incontinence garment. The composition of the disclosure can further be present in a viscous liquid such as a lotion, cream, or medicament as well.

It is known that organic aldehydes can be converted into acetals by reacting with alcohols. Those acetals can be hydrolyzed under acidic condition to convert back into alcohol and aldehyde. The chemistry has been proposed to stabilize and release aldehyde-based fragrances, but all the products reported are relatively hydrophobic. Release of the aldehyde fragrances through hydrolysis of hydrophobic acetals requires the assistance of one or more organic solvents to improve water solubility. To obtain a reasonable release rate using those, an acidic medium is normally needed. Furthermore, those acetals are very often liquid and volatile themselves. Therefore, standard acetals are not ideal for stabilization and controlled release of aldehyde-based actives. Finally, it is not known to use stimuli-sensitive encapsulation systems in conjunction with acetal-based pro-actives to achieve stabilization and controlled release of actives triggered by multiple triggers or stimuli.

One aspect of this disclosure is organic salt-based acetals that can be either semi-solid, solid, or liquid, for modification and controlled release of aldehyde-based actives. The general molecular structure of the acetals is shown below, in which active aldehyde R—CHO can be released upon hydrolysis. R¹R²R³—C and R⁴R⁵R⁶—C are carrier moieties. R¹R²R³—C and R⁴R⁵R⁶—C will be converted into R¹R²R³—COH and R⁴R⁵R⁶—COH after release of aldehyde actives. Either R¹R²R³—C or R⁴R⁵R⁶—C or both have one or more charged groups (positively charged, negatively charged, or both). R¹, R², R³, R⁴, R⁵, and R⁶ can have organic or inorganic moieties. R¹, R², R³, R⁴, R⁵, and R⁶ can be the same or different. The salt-based acetals contain at least one charged group with its counter ion. The charged group(s) is located in the portion of the carrier of the acetals. The charged groups help improve water-solubility of the acetals and reduce their volatility. The improved water solubility is important for rapid release of the actives by water.

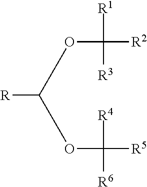

### General Molecular Structure of Acetal Proactives

Examples of the charged groups in the pro-active acetals include, but are not limited to, negatively charged sulfonate, phosphate groups and carboxylic acid group (under basic pH), positively charged amino (under acidic condition) or quaternary ammonium groups.

Examples of aldehyde-based actives include, but are not limited to, fragrances, antimicrobial agents, skincare actives, antibiotics, and hormones.

Two specific salt-based pro-active acetals for volatile fragrances:

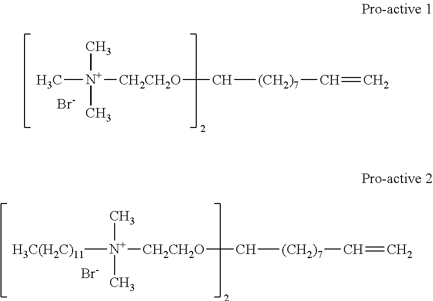

Another aspect of the disclosure is a composition that includes at least one of the salt-based acetal pro-actives and at least one of the stimuli-sensitive polymers. The stimuli-sensitive polymers can respond to different stimuli such as water, body fluids such as urine and nasal secretion, pH, temperature, and light.

Another aspect of the disclosure is a product that includes the salt-based acetal pro-actives or a composition that includes the pro-active. Examples of such products include, but are not limited to, absorbent products, tissues, medical devices, face-masks, lotions, creams, and wipes.

The functional aldehyde-based active of the composition can be a fragrance, an antioxidant, an antimicrobial or antibacterial agent, or a skin-repairing agent. The functional active has at least one aldehyde group in its molecular structure. The functional active is converted into an organic salt-based acetal. The rationale for converting the active (R—CHO) into an organic salt-based acetal is to modify the properties of the active. There are several properties of actives that can be modified by this structural change, such as volatility (and the consequential difficulty in storage, handling, and processing). The acetal form of the active would be nonvolatile. The property of oxidation can also be controlled by conversion of a material into the organic salt-based acetal form.

Antioxidants and skin-repair agents (such as retinaldehyde) can also be placed in a more stable form when converted into an organic salt-based acetal. Some actives demonstrate poor permeability (such as retinaldehyde) through biological barriers such as skin. The organic salt-based acetal form of such actives can be used to balance the hydrophilicity/hydrophobicity of the active to improve skin permeability. The organic salt-based acetal form can also be used to control the release rate of an active.

R is an organic moieties such as alkyl or its derivatives with functional groups. R—CHO is an active. X— is an counter ion. The acetal bond can be hydrolyzed under mild conditions to generate R—CHO upon exposure to moisture or aqueous media or hydrolases. The (R) group is a radical of the functional active, such as the radical of a volatile aldehyde-based fragrance. The (R) group includes components having the desired functionality. One or more of the R¹, R², R³, R⁴, R⁵, and R⁶ groups have at least one charged moieties. It should be recognized that the more hydrophobic the R¹, R², R³, R⁴, R⁵, and R⁶ groups are, the more difficult also for the organic salt-based acetal to solubilize in water as well as undergo hydrolysis. Further, the more hydrophilic the R group, the less stable the organic salt-based acetal with associated (R) group is, in the sense that it is more likely that vapor/humidity in the air alone will cause the disassociation of the (R—CHO) group (such as aldehyde fragrance) from the organic salt-based acetal molecule. Further, if the organic salt-based acetal is too hydrophobic; that is, if it includes large hydrophobic groups in the R¹, R², R³, R⁴, R⁵, and R⁶ groups, the more likely that it will not be water soluble, or less so. The (R—CHO) group can include radicals of nonfragrance functional components, such as retinaldehyde, which is attached at the acetal linkage.

As noted, it is desirable that the organic salt-based acetal is not too large such that it cannot be easily solubilized in water or aqueous media. In one aspect, the functional active (radical of the fragrance aldehyde aspect) of the (R) group is selected from the fragrance group including nonadyl, anisaldehyde, vanillin, 10-undecenal, heptanal, 4-decenal, benzaldehyde, phenylacetaldehyde, citral, citronellal, cinnamaldehyde. In another desirable aspect, the active group (R) on the organic salt-based acetal is derived from retinaldehyde.

In general, organic salt-based acetals, their derivatives, and their preparation are known, and as such, the synthesis steps of particular organic salt-based acetals with radical groups (such as fragrance radicals) will not be further delineated. Examples of relatively smaller organic salt-based acetal molecules with attached fragrance radicals (radical groups of volatile aldehydes) can also be found. It has now been found, however, that such chemistry is particularly well suited as a base chemistry for an active delivery formulation on various substrates and absorbent articles and in various formulations, particularly if such organic salt-based acetals are limited in size and do not severely impact absorbency pathways either as a result of their level of hydrophobicity or particular placement on a substrate or within an absorbent article.

Desirably, in one aspect, the organic salt-based acetal with attached chemical active is present in the composition (such as a coating) in an amount between about 0.1 and 30% by weight, alternatively, between about 0.5 to about 15 weight %, further alternatively, between about 1 to 10 weight %. The weight percentages given for this and further composition components are based on the total weight of the dried composition. It should be recognized that some compositions of the disclosure will initially utilize organic solvents for initial application of the composition to substrates, although such solvents are contemplated as being dried off during manufacture. Further, it is contemplated that such compositions can also be applied to substrates as hot melted coatings.

As a result of the moisture/aqueous media sensitivity of certain organic salt-based acetals as noted above, for those organic salt-based acetals with hydrogen or lower alkyl R groups, it can be desirable to insulate the organic salt-based acetal from moisture and aqueous medium before use, so as to delay release of functional active chemicals from hydrolysis. This delay of functional active release can be accomplished by encapsulating the organic salt-based acetal in an encapsulating polymer matrix. The encapsulating matrix can be either dissolved/degraded by aqueous media or can be swollen by water to expose the organic salt-based acetals to water for hydrolysis under various conditions.

The encapsulation chemistry of the present composition desirably is triggerable by the occurrence of one or more stimuli to free up the organic salt-based acetal protected by the encapsulation chemistry. Such encapsulation chemistry (encapsulation polymer matrix) can be in the form of a continuous cover of polymer/particles, microparticles, nanoparticles, encapsulation polymer coating sheets, films, fibers, laminates, foams, pastes, tablets, or suppositories. In such an instance, encapsulating polymers can act as the encapsulation matrix in which the organic salt-based acetals or organic salt-based acetal derivatives are embedded throughout the whole polymer matrix. Alternatively, such encapsulation chemistry can be a shell of a core/shell configuration, such that an encapsulating polymer shell surrounds the organic salt-based acetal core. Such encapsulation chemistry desirably is triggered by pH changes in the environment, but can also be triggered by enzymatic changes, solubility change, changes in temperature via thermogels, changes in ionic concentration, and changes in ligand chemistry.

There are a number of polymers that can be used to achieve this protection through encapsulation of the organic salt-based acetals and derivatives of organic salt-based acetals. For example, in one aspect, dextrans and derivatives can be blended with organic salt-based acetals or organic salt-based acetal derivatives to form films. Upon contact with an aqueous medium, the dextran and derivatives can be dissolved and the organic salt-based acetal then exposed to water for hydrolysis, thereby releasing the functional active.

Environmentally triggerable encapsulation materials that are triggerable upon specific environmental stimuli can include copolymers of methacrylic acid and methyl methacrylate, which are sensitive to basic aqueous solutions. Such materials are available under the trade designations EUDRAGIT S-100, available from Degussa. Alternatively, a copolymer based on dimethylaminoethyl methacrylate, butyl methacrylate, and methyl methacrylate that is sensitive to acidic aqueous solutions can be used. Such materials are available under the trade designation EUDRAGIT E-100 for example, from Degussa. Further encapsulation materials can include vinylpyrrolidone/vinyl acetate copolymers that are sensitive to neutral aqueous solutions. For example, such are available under the trade designations PVP/VA 1-335 from Ashland/ISP.

Certain polymers that are sensitive to basic aqueous solutions, such as copolymers of methacrylic acid and methyl methacrylate, are particularly effective encapsulation chemistry for use with organic salt-based acetals to minimize water sensitivity and solubility under certain pH. For example, when such organic salt-based acetal and polymer films are exposed to neutral water, little active is released. Upon exposure to alkaline aqueous solutions (such as of pH 9), however, such actives are steadily released. Similar performance can be demonstrated for films made from such organic salt-based acetals and a copolymer based on dimethylaminoethyl methacrylate, butyl methacrylate, and methyl methacrylate when exposing such films to first neutral aqueous solutions, and then to slightly acidic solutions (pH 5.5).

By using a composition having encapsulation material that is triggered by a specific change in the environment, such as for example, contact with vaginal fluids that might be excreted from a user with a vaginal infection, or for contact with other basic or slightly basic environments, the first stage trigger can be activated, thereby freeing up potential access to the second stage trigger of the organic salt-based acetal. For example, particular ailments can raise the pH level of vaginal secretions from a normally acidic level to a neutral or slightly alkaline level. Under normal conditions in which pH of such secretions is acidic, such encapsulation chemistry would not be triggered. However, once vaginal fluid of a neutral or slightly alkaline level is introduced to the encapsulation chemistry triggered by a neutral or slightly alkaline environment, the encapsulation chemistry would allow for the release of organic salt-based acetals or organic salt-based acetal derivatives. Upon continued contact of the organic salt-based acetals or organic salt-based acetal derivatives with an aqueous medium, the functional active on the organic salt-based acetal would be released.

Examples of various encapsulation chemistries useful in the disclosure are illustrated below.

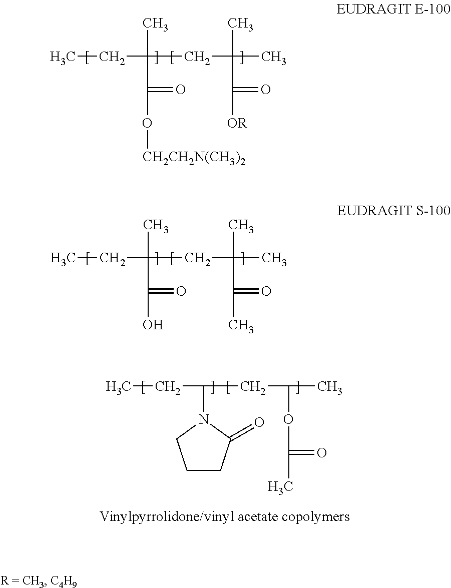

Desirably, for the purposes of this application, the amount of encapsulation chemistry present in the composition is between about 20 and 99.9% by weight. Alternatively, such encapsulation chemistry is present in the composition is between about 40 and 90% by weight. Still in a further alternative aspect, such encapsulation chemistry is present in the composition in an amount of between about 60 and 95% by weight.

The triggerable composition can also contain other components such as solvents, plasticizers, surfactants or wettability agents, pH adjusters, and viscosity enhancers. Based on the substrate or surface on which the composition is to be deposited, or the lotion, cream, or medicament that the composition is to be used in, the composition can require addition of other ingredients to immobilize or adhere the encapsulation and organic salt-based acetal components more securely to the substrate, or in the formulation. The composition can also contain water-miscible or hydrophilic polymers. Furthermore, the composition can also contain other additives to adjust surface tension or other physical and chemical properties. Alternatively, the substrates can be treated with different materials to modify their surface properties before the deposition of the composition to improve the adhesion of the composition. The wettability-enhancing agent can be a single surfactant or a mixture of surfactants. The surfactants can be non-ionic, neutral surfactants, or ionic surfactants. The ionic surfactants can be either positively charged or negatively charged. Examples of non-ionic surfactants include alkyl poly(ethylene oxide) such as copolymers of poly(ethylene oxide) and poly(propylene oxide) (commercially called Poloxamers or Poloxamines), alkyl polyglucosides such as octyl glucoside and decyl maltoside, and fatty alcohols such as cetyl alcohol, oleyl alcohol, cocamide MEA and cocamide DEA. Examples of ionic surfactants include anionic (e.g., based on sulfate, sulfonate, or carboxylate anions) surfactants such as s (SDS), ammonium lauryl sulfate, and other alkyl sulfate salts, Sodium laureth sulfate, also known as sodium lauryl ether sulfate (SLES), Alkyl benzene sulfonate, soaps, and fatty acid salts; and cationic (e.g., based on quaternary ammonium cations) surfactants such as Cetyl trimethylammonium bromide (CTAB) a.k.a. hexadecyl trimethyl ammonium bromide, and other alkyltrimethylammonium salts, Cetylpyridinium chloride (CPC), Polyethoxylated tallow amine (POEA), Benzalkonium chloride (BAC), and Benzethonium chloride (BZT); or Zwitterionic (amphoteric) surfactants such as Dodecyl trigonelline, Dodecyl dimethylamine oxide, Cocamidopropyl trigonelline, and Coco ampho glycinate. Alternatively, the wettability-enhancing agents can also be hydrophilic molecules. The hydrophilic molecules can also be polymers such as polyethylene glycol and its copolymers.

The triggerable composition of the disclosure can be applied to a substrate such as an absorbent article or a layer within an absorbent article by any number of known applications or printing techniques. For example, the triggerable composition of the present disclosure can be deposited on a substrate by various surface deposition or printing methods such as brushing, flexographic printing, gravure roll printing, stamping, screen print, spraying techniques, dip and squeeze, and digital print methods. Further, the composition can be applied in a melt form and allowed to solidify on a treated substrate. As also noted, the composition can be part of a lotion, cream, or medicament as well.

Placement of the triggerable composition can be on any number of substrates. The substrate sheets can, for instance, include nonwoven or woven sheets. Such sheets can include synthetic or natural fibrous materials such as, for example, extruded spunbond, meltblown webs, bonded carded webs, airlaid materials, spun cellulosic, and wool or synthetic yarns. Such sheets can further include cellulosic-based dry or wet laid tissue or paper sheets. Additionally, such substrates can include film or foam sheets, laminates of film, foam and fibrous layers, and laminates of multiple fibrous, film, and foam layers. Such substrates/sheets can be placed as layers within medical or beauty care articles, personal care hygienic articles such as absorbent articles, or can themselves serve as the absorbent article, including as a towel, tissue, or wipe. Further, such triggerable composition can be used as components in lotions, creams, and medicaments, including tablets and suppositories.

Placement of such composition in an article or absorbent article can be across the entire article's longitudinal and transverse or lateral (width) dimensions, or on a layer of an article. Placement can be limited to certain locations within the article, or layer(s) on the article. For example, such composition can be placed at a location specifically designed to contact aqueous-based waste, such as a high-probability soiling area in an article's or layer's central crotch region. Such treated layers can include the topsheet layer, backsheet layer (inner surface), or absorbent core layer. Other interior-positioned layers can also be treated with the coating composition. It can be desirable to limit the placement of the coating formulation to certain locations on an absorbent article that would not directly impact the absorbency pathways of an article, such as on an inside surface of a backsheet layer (as opposed to a topsheet layer or absorbent core layer), or side areas of a topsheet layer, absorbent core layer, or other interior-positioned layer.

### Examples

The following components were blended together to form coating compositions for the purpose of demonstrating the effectiveness of using a two stage triggerable composition, including an encapsulated organic salt-based acetal with functional active, according to the present disclosure. For some applications, those organic salt-based acetals need to be insulated from moisture and water before use. This issue can be solved by encapsulating them in a protecting matrix. The protecting matrix can either be dissolved by aqueous media or can be swollen by water to expose the acetals to water for hydrolysis under various conditions. There are a number of polymers that can be used to achieve the protection while allowing water or moisture to penetrate under various conditions.

1. Acetal of citronellal with ethanol and acetal of vanillin with ethanol were custom-synthesized. Their structures are shown below. The two non-salt acetals were found to be liquid and have similar volatility with the parent citronellal and vanillin. The acetals were embedded in PVP/VA, Eudragit E-100, and Eudragit S-100 by simply dissolving the acetals in the solutions of those polymers. The solutions were then brushed on polyethylene films and air-dried. The parent aldehydes were also embedded in the same ratio in those polymers. Strong smells of both parent aldehydes and acetals could be nasally detected after drying. Those non-salt based acetals cannot effectively reduce the volatility of aldehyde-based fragrances and are not good pro-actives for stabilizing those fragrances.

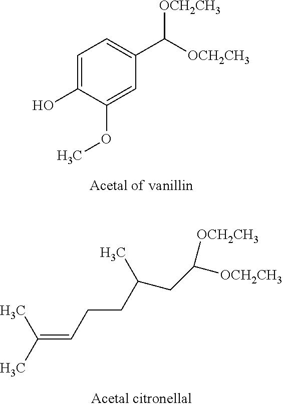 [SMILES: CCOC(OCC)c1ccc(O)c(OC)c1 ; CCOC(CC(C)CCC=C(C)C)OCC]

2. Salt-based acetals pro-active 1 and pro-active 2 as shown above were designed and synthesized. Pro-active 1 has two quarterly ammonium groups in its alcohol-based carriers of short alkyl chain and is substantially water-soluble. Pro-active 2 also has two quarterly ammonium groups in its two long alkyl chains. Both pro-actives were found to be a wax-like solid and are substantially not volatile. Pro-active 2 was found to be significantly less water-soluble than pro-active 1. In dry form, the smell of the two pro-actives is almost non-detectable by a human nose.

3. 100 mg of pro-active 1 was dissolved in 2 ml mixing solvent of acetone and methanol to make a stock solution. 200 microliters of the stock solution were transferred to each of eight vials which were designated as samples 1, 2, 3, 4, 5, 6, 7, and 8, respectively. To sample 8 was further added 10 mg polyacrylic acid (PAA). The solvent was then removed by air-stream. Sample 8 was placed in an oven of 50° C. overnight. Sample 8 in dry form had a scent that was almost non-detectable by human nose. To each of samples 2, 3, 4, 5, 6, and 7 was added with 1 ml water of pH 7, 6, 5, 4, 3, and 2, respectively. Almost immediately the scent of 10-undecenal was detected by nose. The pH of the water was adjusted with lactic acid and sodium hydroxide. To sample 8, addition of pure water results in very strong scent of 10-undecenal, which is much stronger than those samples without PAA.

4. The same experiments were conducted for pro-active 2. All the procedures were the same as experiment 3 except for the replacement of pro-active 1 with pro-active 2. Unlike pro-active1 where a strong scent of 10-undecenal was immediately detected, the release of 10-undecenal from Pro-active 2 was more steady and slower under all the conditions.

5. a. Preparation of prototypes: Three types of VIVA brand paper towel samples were prepared. Sample 1 was prepared by spraying a solution containing 100 mg pro-active 1 and 11 ml acetone on a paper towel and drying at 50° C. in an oven overnight. Sample 2 was prepared by spraying a solution containing 100 mg pro-active 1, 1 g polyacrylic acid, 7 ml acetone, and 3 ml methanol on a paper towel and drying at 50° C. in an oven overnight. Sample 3 was prepared by spraying a solution containing 100 mg pro-active 1, 1 g PVP/VA(I335), 7 ml acetone, 1 ml isopropanol, and 2 ml methanol on a paper towel and drying at 50° C. in an oven overnight.

b. Testing the prototypes: Water was sprayed on a piece of each sample prepared in Example 5. Sample 1 showed a slight scent upon water insult. Sample 2 showed a very strong scent immediately while sample 3 showed moderate level scent immediately upon contact with water.

As described above, in one aspect the present disclosure is directed to incorporating a two-stage triggerable composition into an absorbent article such as a health care product including a garment or bandage, a hygiene product including a tissue or wipe, a skin-contacting beauty product including a facial wrap, or an absorbent consumer/personal care article including a feminine care pad or liner, a baby or child care diaper, or an adult incontinence garment. In particular, the composition is placed on a layer within the article and configured to release a scent, antibacterial agent, skin-repairing agent, antioxidant agent, or other functional active when exposed to a first environmental stimulus followed by contact with an aqueous medium such as urine, menses, vaginal secretions, sweat, mucous, or a loose bowel movement. In one aspect, for instance, the composition is coated as a patch on an individual layer within a diaper, which will be exposed to an aqueous medium following contact with an initial environmental stimulus. The composition, for example, can be coated on a portion of the topsheet layer (user facing surface or garment facing surface), the absorbent core layer (or other internal article layer), or on the inside surface of the backsheet layer. Alternatively, such composition can be disposed on a discrete patch of separate material that functions as a carrier layer, such as, for example, a nonwoven material that includes a user-facing surface. The composition is released upon triggering by an environmental stimulus and contact with an aqueous medium. The two stage triggerable composition can also be made in particulate form and mixed with superabsorbent materials or other absorbent components as a part of an absorbent layer.

As can be seen, controlled release of chemical actives can be achieved in a two-stage process by using a stimulus-sensitive encapsulation chemistry and an aqueous medium-sensitive organic salt-based acetal chemistry in a single composition. Such a composition relies on two different triggering stimuli (such as pH and aqueous medium contact or enzyme and aqueous medium contact) to release an active chemistry, thereby providing stability to functional actives, and control in the graduated release of such actives to the environment or a desired location.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present disclosure. To the extent that any meaning or definition of a term in this written document conflicts with any meaning or definition of the term in a document incorporated by reference, the meaning or definition assigned to the term in this written document shall govern.

While particular aspects of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

We claim:

1. A triggerable composition for two-stage, controlled release of a functional active chemical comprising:
an aldehyde-based functional active incorporated in an organic salt-based acetal that releases the functional active through a hydrolysis reaction upon contact with an aqueous medium; and
an encapsulation material for encapsulating the organic salt-based acetal, the encapsulation material triggerable to release or expose the organic salt-based acetal upon the occurrence of an environmental stimulus.

2. The triggerable composition of claim 1, wherein the organic salt-based acetal has the following molecular structure: 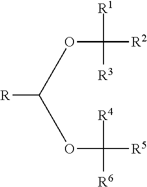
and wherein one or more of the R¹, R², R³, R⁴, R⁵, and R⁶ groups have at least one charged moieties.

3. The triggerable composition of claim 1, wherein the stimulus is selected from the group consisting of presence of water, a pH change, an enzymatic change, a temperature change, an ion concentration change, and a ligand concentration change.

4. The triggerable composition of claim 1, wherein the composition is in a form of particles, microparticles, nanoparticles, fibers, sheet, films, or a combination thereof.

5. The triggerable composition of claim 1, wherein the encapsulation material is triggerable to release or expose the organic salt-based acetal upon the occurrence of a pH change.

6. The triggerable composition of claim 1, wherein the encapsulation material is selected from copolymers of methacrylic acid and methyl methacrylate that are sensitive to basic aqueous solutions, copolymers of dimethylaminoethyl methacrylate, butyl methacrylate, and methyl methacrylate that are sensitive to acidic aqueous solutions, and vinylpyrrolidone/vinyl acetate copolymers that are sensitive to neutral aqueous solutions.

7. The triggerable composition of claim 1, wherein the functional active is selected from the group consisting of a fragrance, an antimicrobial agent, an antioxidant agent, a skin repairing agent, an antifungal agent, a hormone, and a medically active agent.

8. The triggerable composition of claim 7, wherein the functional active is a fragrance having at least one aldehyde group.

9. The triggerable composition of claim 7, wherein the functional active is a skin repairing agent having at least one aldehyde group.

10. An absorbent article including at least one absorbent layer, the absorbent article including a triggerable composition for two-stage, controlled release of a functional active chemical, the triggerable composition comprising:
an organic salt-based acetal of a functional active with at least one aldehyde group configured to release the functional active through a hydrolysis reaction upon contact with an aqueous medium; and
an encapsulation material for encapsulating the organic salt-based acetal including a functional active, the encapsulation material triggerable to release or expose the organic salt-based acetal upon the occurrence of an environmental stimulus.

11. The absorbent article of claim 10, wherein the organic salt-based acetal has the following molecular structure: 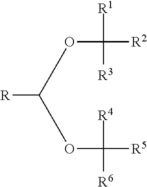
and wherein one or more of the R¹, R², R³, R⁴, R⁵, and R⁶ groups have at least one charged moieties.

12. The absorbent article of claim 10 further comprising a topsheet layer, a backsheet layer, and at least one absorbent core layer, wherein the triggerable composition is included with at least one of the topsheet layer, absorbent core layer, and the backsheet layer.

13. The absorbent article of claim 10, further comprising a carrier layer, wherein the triggerable composition is included with the carrier layer for carrying the triggerable composition within the absorbent article.

14. The absorbent article of claim 10, wherein the absorbent article is selected from the group consisting of feminine care hygiene articles, adult incontinence articles, baby and child care articles, bandages, medical garments, and skin treatment sheets.

15. A viscous liquid comprising:
a triggerable composition for two-stage, controlled release of a functional active chemical, the composition including:
an organic salt-based acetal of a functional active with at least one aldehyde group configured to release the functional active through a hydrolysis reaction upon contact with an aqueous medium, and
an encapsulation material for encapsulating the organic salt-based acetal including a functional active, the encapsulation material triggerable to release or expose the organic salt-based acetal upon the occurrence of an environmental stimulus,
wherein the viscous liquid is a lotion, cream, or medicament.

16. The viscous liquid of claim 15, wherein the organic salt-based acetal has the following molecular structure: 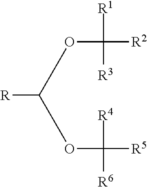
and wherein one or more of the R¹, R², R³, R⁴, R⁵, and R⁶ groups have at least one charged moieties.

17. A triggerable composition for two-stage, controlled release of a functional active chemicals comprising:
an organic salt-based acetal for release of a functional active contained on the organic salt-based acetal, through a hydrolysis reaction upon contact with an aqueous medium, and
an encapsulation material for encapsulating the organic salt-based acetal including a functional active, the encapsulation material triggerable to release or expose the organic salt-based acetal upon the occurrence of an environmental stimulus, wherein the environmental stimulus is a pH change.

18. The composition of claim 17, wherein the stimulus is a pH change from an acidic to neutral or basic environment, and wherein the encapsulation material is selected from the group consisting of copolymers of methacrylic acid and methyl methacrylate that are sensitive to basic aqueous solutions, and vinylpyrrolidone/vinyl acetate copolymers that are sensitive to neutral aqueous solutions.

19. The composition of claim 17, wherein the stimulus is a pH change from a basic to neutral or acidic environment, and wherein the encapsulation material is selected from the group consisting of copolymers of dimethylaminoethyl methacrylate, butyl methacrylate, and methyl methacrylate that are sensitive to acidic aqueous solutions, and vinylpyrrolidone/vinyl acetate copolymers that are sensitive to neutral aqueous solutions.

20. The composition of claim 17, wherein the functional active is a fragrance.
